(19) United States
(12) Patent Application Publication
(10) Pub. No.: US 2022/0287746 A1
(43) Pub. Date: Sep. 15, 2022

(54) BONE FIXATION SYSTEM

(71) Applicant: ATLAS TECHNOLOGIES, LLC, Hewlett, NY (US)

(72) Inventor: David GITLIN, Woodmere, NY (US)

(73) Assignee: ATLAS TECHNOLOGIES, LLC, Hewlett, NY (US)

(21) Appl. No.: 17/828,726

(22) Filed: May 31, 2022

Related U.S. Application Data
(63) Continuation of application No. 16/849,426, filed on Apr. 15, 2020.

(51) Int. Cl. A61B 17/72; A61B 17/68; A61F 2/28

(52) U.S. Cl. CPC A61B 17/7241; A61B 17/683; A61F 2/28; A61F 2002/30604

(57) ABSTRACT

A bone fixation system includes an intramedullary rod, a cage that is configured to receive the intramedullary rod at least partially therethrough, and at least one fixator configured to connect the intramedullary rod to the cage. A method for repairing a bone defect in a patient using the bone fixation system includes implanting the cage into the bone defect, inserting at least a portion of the intramedullary rod into the cage, and securing the cage to the intramedullary rod using the fixator.

### RELATED APPLICATION

This application is a continuation of U.S. patent application Ser. No. 16/849,426, filed Apr. 15, 2020, the disclosure of which is incorporated herein by reference in its entirety.

### FIELD OF THE INVENTION

The invention relates generally to medical devices for use in the surgical repair of a defect or disease in a patient's bone, and more particularly, to a bone fixation system and methods for forming and using same.

### BACKGROUND OF THE INVENTION

As is known to those in the art of orthopedic surgery, cage implants are used in orthopedic surgery to repair various bone defects that are caused by disease, trauma or other causes. For example, a cage implant can be used to fill in a segmental bone defect. These cages are filled with bone graft material (e.g., bone grafts harvested from the patient (i.e., an autograft), from a donor that is the same species as the patient (i.e., an allograft), or from a species that is different than the patient (i.e., a xenograft)), and placed in the surgical site to repair the defect. But cages, by themselves, may be unstable and thus need additional support in order to be securely held in place to immobilize the area of fixation. So a cage may be supplemented by the use of additional implants. For example, an intramedullary rod may be placed through the cage to help stabilize the fixation area.

Intramedullary rods, otherwise simply referred to as “rods” for efficiency, are also used in orthopedic surgery to repair long bone defects and injuries. However, rods, alone, may also create an unstable construct. Therefore, a rod may be supplemented with plate fixation or with a cage. Nonetheless, rod-cage constructs are still believed to have some instability.

Thus, it is desirable to improve rod-cage systems to improve patient outcomes as well as enhance surgeon usability, by coupling the rod to the cage (e.g., via screws). The cage is modular. The rod can also be modular.

### SUMMARY OF THE INVENTION

The present disclosure provides a bone fixation system. The bone fixation system comprises a height-adjustable cage having opposed first and second ends, a wall extending between the first and second ends, a longitudinal axis extending through the first end, second end and wall, a static cage section and at least one dynamic cage section moveable along the longitudinal axis and with respect to the static section; and an intramedullary rod having opposed first and second ends and configured to extend completely through the cage generally along the cage's longitudinal axis. The intramedullary rod is configured to be fixed to the cage with at least one fixator inserted transversely through the rod and the cage, either before or after the cage height is adjusted.

The present disclosure also provides a method for repairing a bone defect in a patient. The method comprises a step of providing a bone fixation system, the bone fixation system including a height-adjustable cage having opposed first and second ends, a wall extending between the first and second ends, a longitudinal axis extending through the first end, second end and wall, a static cage section and at least one dynamic cage section moveable along the longitudinal axis and with respect to the static cage section; and an intramedullary rod having opposed first and second ends and configured to extend completely through the cage generally along the cage's longitudinal axis. The intramedullary rod is configured to be fixed to the cage with at least one fixator inserted transversely through the rod and the cage, either before or after the cage height is adjusted. The method also includes the steps of implanting the cage into the bone defect, inserting the intramedullary rod into the cage and securing the cage to the intramedullary rod using the at least one fixator.

The present disclosure also provides a kit for performing bone fixation surgery. The kit comprises a height-adjustable cage having opposed first and second ends, a wall extending between the first and second ends, a longitudinal axis extending through the first end, second end and wall, a static cage section and at least one dynamic cage section moveable along the longitudinal axis and with respect to the static section; an intramedullary rod having opposed first and second ends and configured to extend completely through the cage generally along the cage's longitudinal axis; and at least one fixator, wherein the rod is configured to be fixed to the cage with the at least one fixator inserted transversely through the rod and the cage, either before or after the cage height is adjusted.

### BRIEF DESCRIPTION OF THE FIGURES

The accompanying figures, which are incorporated in and constitute a part of the specification, illustrate preferred embodiments of the invention and, together with the description, serve to explain the objects, advantages, and principles of the invention. Embodiments of the invention are in no way limited by the following figures:

FIG. 1 is a schematic view of a bone fixation system according to an embodiment of the present invention, as shown assembled;

FIG. 2 is a schematic exploded view of the bone fixation system of FIG. 1;

FIG. 3 is a schematic exploded view of a cage of the bone fixation system of FIG. 1;

FIG. 4 is a schematic environmental view of the bone fixation system of FIG. 1, as implanted in an exemplary long bone;

FIG. 5A is a schematic top view of an embodiment of the bone fixation system of FIG. 1, wherein the cage has a closed, two-piece construction;

FIG. 5B is a schematic top view of an embodiment of the bone fixation system of FIG. 1, wherein the cage has an open, one-piece construction; and

FIG. 6 is a schematic top view of another embodiment of a bone fixation system according to an embodiment of the present invention, as shown assembled.

### DETAILED DESCRIPTION OF THE INVENTION

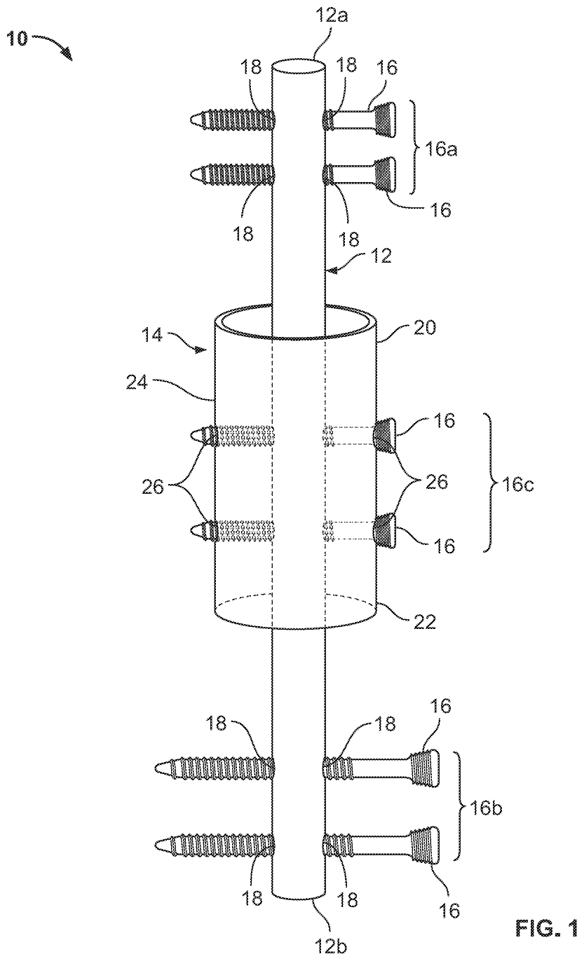

FIGS. 1-2 and 4 illustrate a bone fixation system, or assembly, 10 according to an embodiment of the invention. The bone fixation system 10 is used to repair defects in various long bones, such as, but not limited to, the femur, tibia, fibula, ulna, radius, and humerus. The bone fixation system 10 includes an intramedullary rod, or nail, 12, a cage 14 that is configured to receive the intramedullary rod 12 at least partially therethrough, and transfixion screws 16 that engage both the intramedullary rod 12 and cage 14. These components are first discussed individually below and are then discussed together in the system 10 of the present invention. Each component may be manufactured using known manufacturing methods, including 3-D custom printing, from known biocompatible materials suitable for each component's intended use.

The intramedullary rod 12 is dimensioned for use in various long bone fixation and/or repair surgeries as is well known in the art. The intramedullary rod 12 is used to create stability, enable fixation, and maintain skeletal length and soft tissue tension, thereby ultimately facilitating mobilization and rehabilitation.

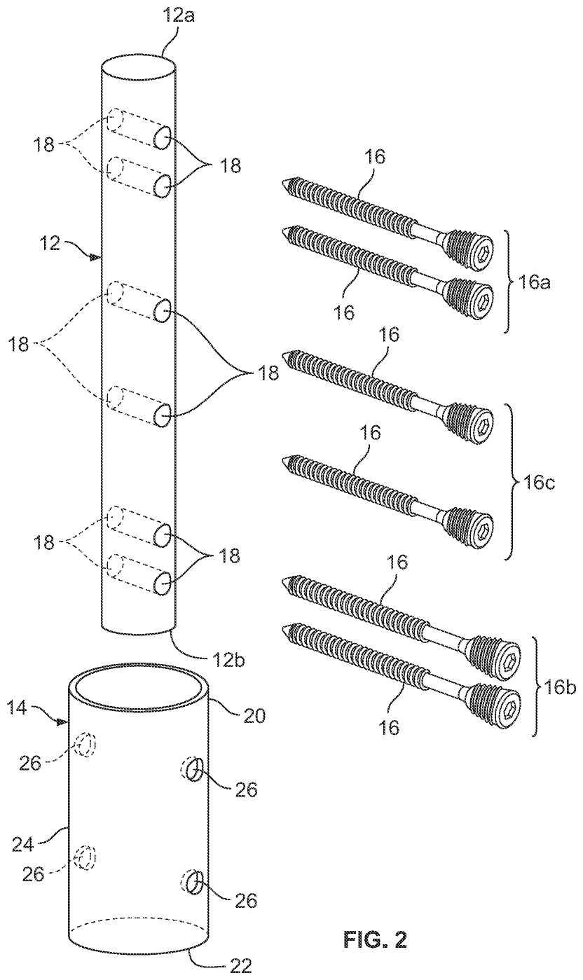

The intramedullary rod 12 includes opposed first and second ends 12a, 12b. The ends 12a, 12b are shown as being blunt/open in FIGS. 1 and 2. However, in one embodiment, it is contemplated that one of the ends 12a, 12b has a bullet nose, to facilitate insertion into the bone canal.

The intramedullary rod 12 also includes a plurality of threaded holes 18 that are dimensioned to receive the transfixion screws 16 therethrough (see FIG. 2). The transfixion screws 16 are further discussed below. In various embodiments, the intramedullary rod 12 includes any number of threaded holes 18, and the threaded holes may be spaced at any interval/distance from each other. In other embodiments, the holes 18 are not threaded. In one such embodiment, the transfixion screws 16 may be secured in the non-threaded holes 18 by an interference fit.

With continued reference to FIGS. 1 and 2, the cage 14 is configured to receive bone graft material (i.e., autograft bone, allograft bone, or xenograft bone, as discussed above) or artificial material, or any combination of both, in order to fill and repair a segmental bone defect in a patient. The cage 14 is configured to receive the intramedullary rod 12 at least partially therethrough (see FIG. 1). In various embodiments, the cage 14 is elongated and has a shape and cross section that enables it to receive the intramedullary rod 12 therein. For example, the cage may be cylindrical, pyramidal or oblong.

In an embodiment, the cage 14 includes opposed first and second ends 20, 22 and a cylindrical wall 24 that extends between the ends 20, 22. In other embodiments, the wall 24 of the cage 14 may have different shapes as are well known in the art. The wall 24 includes one or more holes 26 that are dimensioned to receive the transfixion screws 16 therethrough. In one embodiment, the holes 26 are threaded, and threadably engage the transfixion screws 16. In other embodiments, the holes 26 are not threaded. In one such embodiment, the transfixion screws 16 may be secured in the non-threaded holes 26 by an interference fit. The transfixion screws 16 are further discussed below.

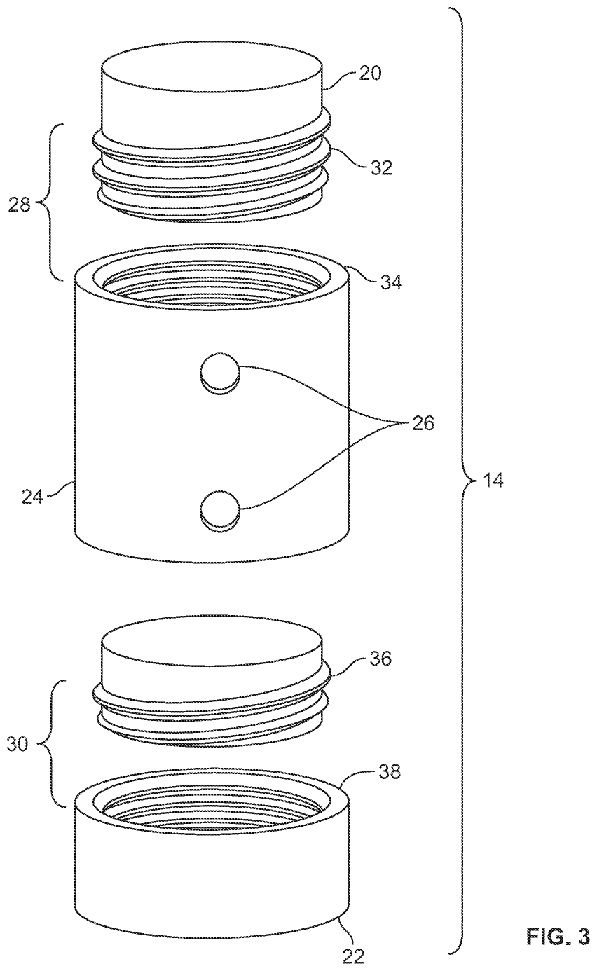

Reference is now made to FIG. 3, which illustrates an embodiment of the cage 14 that is modular, and includes means for adjusting its length, depending on the size of the bone defect that is being repaired. In an embodiment, the cage 14 has a first threaded assembly 28 proximate the first end 20, and a second threaded assembly 30 proximate the second end 22. The threaded assemblies 28, 30 are used to adjust the length of the cage 14 before it is placed into the bone defect and secured to the rod 12.

In various embodiments, the first threaded assembly 28 includes a first outer threaded portion (i.e., having outer threads) 32, and a separate, first inner threaded portion (i.e., having inner threads) 34 that threadably cooperates with the first outer threaded portion. In the embodiment of FIG. 3, the first outer threaded portion 32 is closer to the first end 20, and the first inner threaded portion 34 is farther from the first end 20. In other embodiments, the first outer and first inner threaded portions 32, 34 are in the reverse position: the first inner threaded portion 34 is closer to the first end 20, and the first outer threaded portion 32 is farther from the first end 20.

In various embodiments, the second threaded assembly 30 includes a second outer threaded portion (i.e., having outer threads) 36, and a separate, second inner threaded portion (i.e., having inner threads) 38 that threadably cooperates with the second outer threaded portion 36. Referring again to the embodiment of FIG. 3, the second inner threaded portion 38 is closer to the second end 22, and the second outer threaded portion 36 is farther from the second end 22. In other embodiments, the second outer and second inner threaded portions 38, 36 are in the reverse position: the second outer threaded portion 36 is closer to the second end 22, and the inner threaded portion 38 is farther from the second end 22.

In other embodiments, the cage 14 only includes one threaded assembly. For example, the cage 14 includes either the first threaded assembly 28, or the second threaded assembly 30, but not both.

In various other embodiments, the first end 20, second end 22 and cylindrical wall 24 of the cage 14 are secured to each other using different types of connections/connectors, including, but not limited to, other types of threaded connections, press-fit connections, and combinations of such connections.

In various embodiments, the first end 20, second end 22 and cylindrical wall 24 of the cage 14 have different shapes and/or orientations, including, but not limited to, being distally angled or slanted, in order to create a more customized fit into a patient's segmental defect.

In various embodiments, the first end 20 and/or second end 22 have different types of borders/edges, including, but not limited to, serrated or “toothed” edges.

In various embodiments, the first end 20 and/or second end 22 also have holes for receiving transfixion fixators, to further facilitate fixation/attachment to the intramedullary rod 12 and/or the patient's bone.

In the illustrated embodiment, the first end 20, second end 22 and cylindrical wall 24 are hollow, i.e., do not contain any substance(s). In various other embodiments, the first end 20, second end 22 and/or cylindrical wall 24 are at least partially filled with one or more materials for providing more porous bulk, including, but not limited to metals and plastics.

In an alternate embodiment, the modular cage 14 includes two cooperating components, or “halves”. One such cage is disclosed in U.S. Pat. No. 5,665,122 to Kambin. In another alternate embodiment, the modular cage 14 is a laterally expandable C-shaped cage, as disclosed in U.S. Pat. No. 8,043,376 to Falahee.

With continued reference to FIGS. 1 and 2, the plurality of transfixion screws 16 of the bone fixation system 10 are dimensioned to threadably engage the threaded holes 18 of the intramedullary rod 12 and/or the threaded holes 24 of the cage 14. In an embodiment, a first plurality 16a of transfixion screws 16 engage the first end 12a of the rod 12 and surrounding bone, a second plurality 16b of transfixion screws 16 engage the second end 12b of the rod 12 and surrounding bone, and a third plurality 16c of transfixion screws 16 engage the wall 24 of the cage 14 and portions of the intramedullary rod 12 between its first and second ends 12a, 12b, so as to secure the intramedullary rod 12 within the cage 14, and to the bone.

In an embodiment, the first, second and third pluralities of transfixion screws 16a, 16b and 16c each include two transfixion screws 16. In other embodiments, a single transfixion screw 16 engages the wall 24 of the cage 14 and the intramedullary rod 12 to secure the intramedullary rod 12 within the cage 14, rather than the third plurality 16c. In other embodiments, the first and/or second pluralities 16a, 16b are also replaced with a single transfixion screw 16. In other embodiments, various combinations of one, two or more transfixion screws 16 are used.

In various embodiments, the transfixion screws 16 of the first, second and third pluralities 16a, 16b and 16c have different dimensions. As shown in FIGS. 1 and 2, the transfixion screws 16 of the third plurality 16c are longer than those of the first and second pluralities 16a and 16b. In other embodiments, the transfixion screws 16 of the first, second and third pluralities 16a, 16b and 16c have the same dimensions. The dimensions of the corresponding threaded holes 18 match those of the respective transfixion screws 16.

In various embodiments, the transfixion screws 16 include partial threading for threadably engaging one element (i.e., the cage 14 or the intramedullary rod 12) but slidingly engaging the other element. Other configurations of the transfixion screws 16 are also possible, including various types of threading, lengths, and head sizes and shapes.

In other embodiments, other types of transfixion fixators are used instead of the transfixion screws 16. Such fixators include, for example, rivets, clamps and other transfixion fixators that are known in the art.

In use, a surgeon determines the dimensions of the bone defect, and based on such data, ascertains the dimensions of the components of the bone fixation system 10 (i.e., the intramedullary rod 12, cage 14 and transfixion screws 16) to be used. If an adjustable cage is used, the length of the cage 14 is adjusted to optimally engage with the patient's bone and defect therein. In an embodiment, the length of the cage 14 is adjusted by threadably rotating the first outer threaded portion 32 relative to the first inner threaded portion 34, thereby lengthening or shortening the first threaded assembly 28. In an embodiment, the length of the cage 14 is adjusted by threadably rotating the second outer threaded portion 36 relative to the second inner threaded portion 38, thereby the lengthening or shortening the second threaded assembly 30. In an embodiment, the length of the cage 14 is adjusted by lengthening or shortening both the first threaded assembly 28 and the second threaded assembly 30, as described above.

Whether a singular static cage or a modular cage is used, after the cage 14 has been matched to the bone defect, it is implanted into the patient. In an embodiment, bone graft material is placed inside of the cage 14 prior to its implantation. Following the implantation of the cage 14 into the bone defect, the intramedullary rod 12 is inserted into the cage 14. For example, the second end 12b of the intramedullary rod 12 is introduced into the interior of the cage 14 at the first end 20 thereof, and extended through the cage 14 until it exits from the second end 22 thereof. Once the intramedullary rod 12 is placed through/within the cage 14, the transfixion screws 16 of plurality 16c are inserted into and threadably engaged within the respective threaded holes 26 of the cage 14 and the corresponding threaded holes 18 of the intramedullary rod 12, so as to secure the cage 14 to the intramedullary rod 12. The transfixion screws 16 of pluralities 16a and 16b are then inserted into and threadably engaged within the threaded holes 18 at the first and second ends 12a, 12b of the intramedullary rod 12 in order to further secure the intramedullary rod 12/cage 14 assembly in place. The transfixion screws 16 of pluralities 16a and 16b also engage the surrounding bone into which the system 10 is implanted.

In an embodiment, an aiming jig that is used to place the intramedullary rod 12 is modified to engage the cage 14 in order to properly guide the transfixion screws 16 into their respective threaded holes 26.

As shown in FIGS. 1 and 2, the transfixion screws 16 engage the intramedullary rod 12 and cage 14 so as to be perpendicular to a longitudinal axis of the intramedullary rod 12. In other embodiments, such as that shown in FIG. 6, one or more of the transfixion screws 16 engages the intramedullary rod 12 and/or cage 14 at an angle with the longitudinal axis of the intramedullary rod 12.

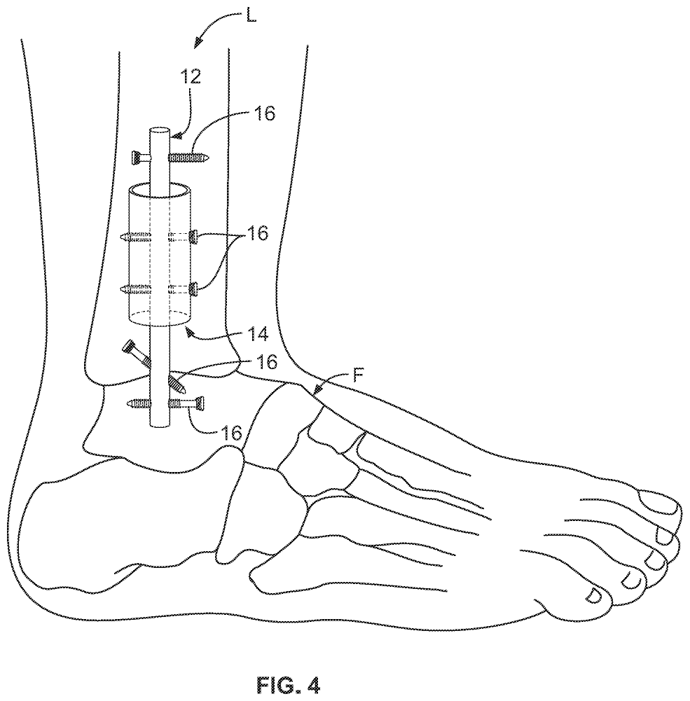

FIG. 4 shows an exemplary placement of the bone fixation system 10 as implanted in a lower leg bone L (i.e., a tibia or fibula) and foot bone F of a patient. The transfixion screws 16 secure the intramedullary rod 12 to the cage 14 and/or the adjacent bone tissue of the lower leg bone L or foot bone F.

The bone fixation system 10 provides a union between the intramedullary rod 12 and the cage 14, thereby increasing the stability of the implantable construct used to repair the bone defect, leading to an improved patient outcome.

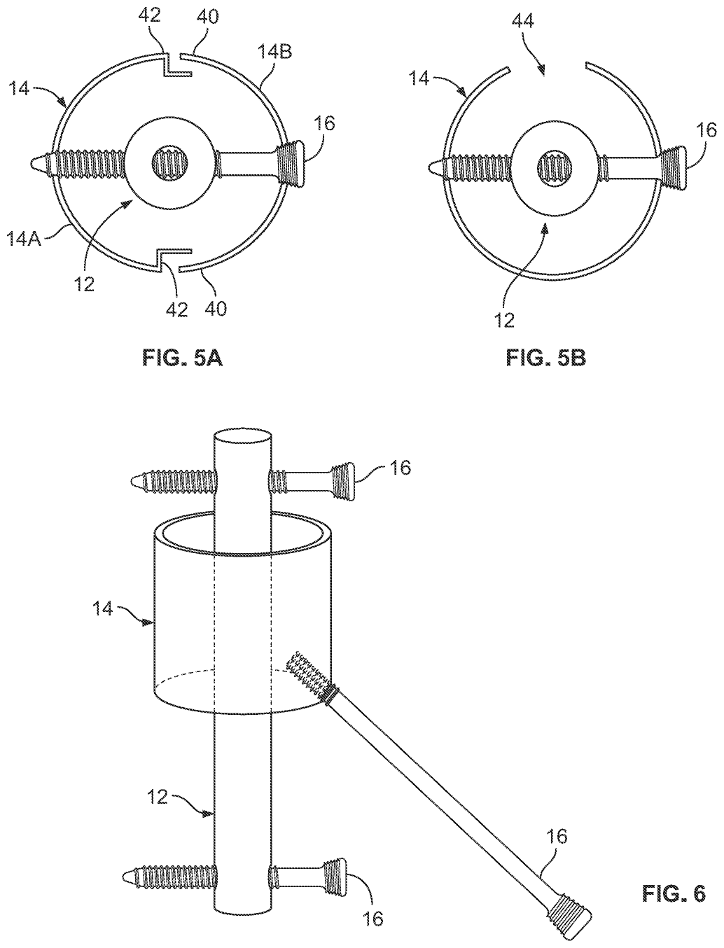

FIGS. 5A and 5B show two alternate embodiments of the cage 14. In FIG. 5A, the cage 14 has a closed configuration, and includes two interconnecting pieces, or halves, 14a and 14b. In the embodiment shown, the half 14b has uncrimped end edges 40 and the half 14a has crimped end edges 42 that are configured to connectively engage the uncrimped end edges 40, so as to secure halves 14a, 14b to each other in forming the cage 14. Other ways of connecting the halves 14a, 14b to each other may also be used. The use of a two-piece cage 14 facilitates the assembly of the bone fixation system 10 during surgery. FIG. 5B shows a cage 14 having a one-piece construction and an open configuration that includes an opening 44. The use of an open cage 14 also facilitates the assembly of the bone fixation system 10 during surgery.

Modifications, additions, or omissions may be made to the systems, apparatuses, and methods described herein without departing from the scope of the disclosure, and are expressly contemplated herein. For example, the components of the systems and apparatuses may be integrated or separated. Moreover, the operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set.

Other embodiments of the invention will be apparent to those skilled in the art from consideration of the specification and practice of the invention disclosed herein. Although specific advantages have been enumerated above, various embodiments may include some, none, or all of the enumerated advantages. It is intended that the embodiments described above be considered as exemplary only, with a true scope and spirit of the invention being indicated by the appended claims. Moreover, none of the features disclosed in this specification should be construed as essential elements, and therefore, no disclosed features should be construed as being part of the claimed invention unless the features are specifically recited in the claims. In addition, it should be understood that any of the features disclosed on any particular embodiment may be incorporated in whole or in part on any of the other disclosed embodiments.

In any interpretation of the claims appended hereto, it is noted that no claims or claim elements are intended to invoke or be interpreted under 35 U.S.C. 112(f) unless the words “means for” or “step for” are explicitly used in the particular claim.

In general, any combination of disclosed features, components and methods described herein is possible. Steps of a method can be performed in any order that is physically possible.

All cited references are incorporated by reference herein.

I claim:

1. A bone fixation system, comprising:
a height-adjustable cage having opposed first and second ends, a wall extending between the first and second ends, a longitudinal axis extending through the first end, second end and wall, a static cage section and at least one dynamic cage section moveable along the longitudinal axis and with respect to the static section; and
an intramedullary rod having opposed first and second ends and configured to extend completely through the cage generally along the cage's longitudinal axis;
wherein the intramedullary rod is configured to be fixed to the cage with at least one fixator inserted transversely through the rod and the cage, either before or after the cage height is adjusted.

2. The bone fixation system of claim 1, wherein the at least one fixator includes at least one transfixion screw, the static cage section having at least one hole dimensioned to receive the at least one transfixion screw therethrough, and the intramedullary rod having at least one hole dimensioned to receive the at least one transfixion screw therethrough, wherein the at least one transfixion screw is configured to securely engage the at least one hole of the intramedullary rod and the at least one hole of the static cage section, to secure the cage to the intramedullary rod.

3. The bone fixation system of claim 2, wherein the at least one transfixion screw includes threading along at least a portion thereof, and the at least one hole in the wall of the static cage section is threaded so as to threadably engage the at least one fixation screw, and wherein the at least one hole in the intramedullary rod is threaded so as to threadably engage the at least one fixation screw.

4. The bone fixation system of claim 1, wherein the at least one dynamic cage section includes a first dynamic cage section proximate the first end of the cage and a second dynamic cage section proximate the second end of the cage.

5. The bone fixation system of claim 1, wherein the at least one dynamic cage section includes at least a first threaded assembly proximate the first end of the cage.

6. The bone fixation system of claim 5, wherein the first threaded assembly includes a first outer threaded portion having outer threads, and a separate, first inner threaded portion having inner threads, the first inner threaded portion configured to threadably cooperate with the first outer threaded portion.

7. The bone fixation system of claim 6, wherein the at least one dynamic cage section further includes a second threaded assembly proximate the second end of the cage.

8. A method for repairing a bone defect in a patient, comprising:
providing a bone fixation system, the bone fixation system including
a height-adjustable cage having opposed first and second ends, a wall extending between the first and second ends, a longitudinal axis extending through the first end, second end and wall, a static cage section and at least one dynamic cage section moveable along the longitudinal axis and with respect to the static cage section; and
an intramedullary rod having opposed first and second ends and configured to extend completely through the cage generally along the cage's longitudinal axis;
wherein the intramedullary rod is configured to be fixed to the cage with at least one fixator inserted transversely through the rod and the cage, either before or after the cage height is adjusted
implanting the cage into the bone defect;
inserting the intramedullary rod into the cage; and
securing the cage to the intramedullary rod using the at least one fixator.

9. The method of claim 8, wherein the at least one fixator includes at least one transfixion screw, the static cage section having at least one hole dimensioned to receive the at least one transfixion screw therethrough, and the intramedullary rod having at least one hole dimensioned to receive the at least one transfixion screw therethrough, wherein the at least one transfixion screw is configured to securely engage the at least one hole of the intramedullary rod and the at least one hole of the static cage section, to secure the cage to the intramedullary rod to facilitate performance of the securing step.

10. The method of claim 9, wherein the at least one transfixion screw includes threading along at least a portion thereof, and the at least one hole in the wall of the cage is threaded so as to threadably engage the at least one transfixion screw as part of the securing step.

11. The method of claim 8, further comprising adjusting the length of the cage to optimally fit into the bone defect.

12. The method of claim 11, wherein the at least one dynamic cage section includes at least a first threaded assembly proximate the first end of the cage, the first threaded assembly including a first outer threaded portion having outer threads, and a separate, first inner threaded portion having inner threads, the first inner threaded portion configured to threadably cooperate with the first outer threaded portion, and wherein the length adjusting step includes threadably rotating the first outer threaded portion relative to the first inner threaded portion to lengthen or shorten the first threaded assembly.

13. The method of claim 12, wherein the at least one dynamic cage section further includes a second threaded assembly proximate the second end of the cage.

14. The method of claim 8, further comprising a step of placing bone graft material inside of the cage prior to the implantation step.

15. The method of claim 8, further comprising a step of securing the bone fixation system to the patient's bone proximate the defect therein by use of the at least one fixator.

16. A kit for performing bone fixation surgery comprising:
a height-adjustable cage having opposed first and second ends, a wall extending between the first and second ends, a longitudinal axis extending through the first end, second end and wall, a static cage section and at least one dynamic cage section moveable along the longitudinal axis and with respect to the static section; and
an intramedullary rod having opposed first and second ends and configured to extend completely through the cage generally along the cage's longitudinal axis; and
at least one fixator, wherein the rod is configured to be fixed to the cage with the at least one fixator inserted transversely through the rod and the cage, either before or after the cage height is adjusted.

17. The kit of claim 16, wherein the at least one fixator includes at least one transfixion screw, the static cage section having at least one hole dimensioned to receive the at least one transfixion screw therethrough, and the intramedullary rod having at least one hole dimensioned to receive the at least one transfixion screw therethrough, wherein the at least one transfixion screw is configured to securely engage the at least one hole of the intramedullary rod and the at least one hole of the static cage section, to secure the cage to the intramedullary rod.

18. The kit of claim 17, wherein the at least one transfixion screw includes threading along at least a portion thereof, and the at least one hole in the wall of the static cage section is threaded so as to threadably engage the at least one fixation screw, and wherein the at least one hole in the intramedullary rod is threaded so as to threadably engage the at least one fixation screw.

19. The kit of claim 16, wherein the at least one dynamic cage section includes a first dynamic cage section proximate the first end of the cage and a second dynamic cage section proximate the second end of the cage.

20. The kit of claim 16, wherein the at least one dynamic cage section includes at least a first threaded assembly proximate the first end of the cage.
